(19) United States
(12) Patent Application Publication
(10) Pub. No.: US 2025/0322904 A1
(43) Pub. Date: Oct. 16, 2025

(54) SYSTEM AND METHOD FOR GENERATING THERMOSTABLE VARIANTS OF A PROTEIN

(71) Applicant: Quantiphi, Inc., Marlborough, MA (US)

(72) Inventors: Dagnachew Birru, Marlborough, MA (US); Meghana Veeramalla, Mumbai (IN); Pooja Kesari, Mumbai (IN); Vishal Pagidipally, Toronto (CA)

(21) Appl. No.: 19/251,338

(22) Filed: Jun. 26, 2025

(51) Int. Cl. G16B 15/20; G16B 30/10; G16B 40/00

(52) U.S. Cl. CPC G16B 15/20; G16B 30/10; G16B 40/00

(57) ABSTRACT

A system and method for generating thermostable variants of a protein is disclosed. The system receives a three-dimensional structure of a target protein and identifies mutable regions, including solvent-exposed residues and loop regions. Conserved and active site residues are excluded from mutation through a fixed-position mask. A message-passing neural network (MPNN) generates mutant sequences at unmasked positions, executed under multiple temperature parameters. Design scores based on Shannon entropy and log probability are computed, and high-confidence variants are selected. Predicted structures for selected variants are evaluated using structural and sequence-based features to compute stability scores. A ranked list of thermostable variants is generated. Top candidates undergo molecular dynamics simulations to compute dynamic metrics such as RMSD, radius of gyration, SASA, and ddG, and are re-ranked accordingly. The system enables accurate, constraint-driven protein design with high structural and functional fidelity, suitable for industrial and therapeutic applications.

### TECHNICAL FIELD

The present technology relates to computational protein engineering, and more specifically, to systems and methods for generating thermostable variants of a protein using machine learning models, structural analysis, and molecular dynamics simulations.

### BACKGROUND

The following description of related art is intended to provide background information pertaining to the field of the disclosure. This section may comprise certain aspects of the art that may be related to various features of the present disclosure. However, it should be appreciated that this section be used only to enhance the understanding of the reader with respect to the present disclosure, and not as admissions of prior art.

Proteins are central to a wide range of industrial, pharmaceutical, and biochemical processes. In many industrial applications, including pharmaceutical manufacturing and chemical synthesis, proteins, often in the form of enzymes, are required to operate under elevated temperatures. However, most naturally occurring proteins tend to denature or lose their functional conformation under thermal stress, limiting their effectiveness and stability in such environments.

To overcome this challenge, protein engineering has emerged as a discipline focused on enhancing the desirable properties of proteins, including their thermostability, without compromising their biological function. Historically, techniques such as directed evolution and rational mutagenesis have been used to explore possible protein variants. While the existing techniques may produce functional and stable mutants, they are often labor-intensive, time-consuming, and heavily reliant on trial-and-error experimentation.

In recent years, computational methods have been introduced to address these limitations. Various machine learning models and structure prediction tools have been used to predict the impact of mutations on protein stability. However, most existing solutions are limited in scope, as these solutions either rely solely on sequence-based analysis or only partially incorporate structural context. Additionally, many such solutions permit mutations across the entire protein sequence, including conserved regions and active sites, potentially disrupting protein folding and function.

The existing techniques typically lack a principled strategy for identifying and isolating mutation-tolerant regions, such as solvent-exposed residues and flexible loop regions, while preserving critical conserved residues and functional sites. Moreover, in most cases, mutant evaluation ends with the prediction of static structures, without examining the dynamic behavior of the protein under thermal conditions. This omission may result in inaccurate assessments of how the mutations impact the protein's stability and integrity during real-world operation.

There is, therefore, a need for an improved system and method that may utilize a machine learning model such as a message-passing neural network (MPNN) for generating thermostable variants of the protein, thereby reducing experimental burden, preserving protein function, and accelerating the thermostable protein variants suitable for industrial or therapeutic use.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect of the present disclosure, the computer-implemented method for generating thermostable variants of a protein is disclosed. The method includes receiving, by at least one processor, a three-dimensional structure of a target protein, wherein a plurality of mutable regions is identified within the three-dimensional structure, and wherein the plurality of mutable regions comprises solvent-exposed residues and loop regions. The method further includes performing, by the at least one processor, a multiple sequence alignment of the target protein to identify a plurality of conserved residues in the target protein. The method further includes generating, by the at least one processor, a fixed-position mask based on the plurality of conserved residues and a plurality of active site residues. The fixed-position mask defines a set of excluded residues that are not subject to mutation. The method further includes generating, by the at least one processor, a plurality of mutant protein sequences using a message-passing neural network by introducing mutations at unmasked positions within the plurality of mutable regions. The method further includes computing, by the at least one processor, a design score for each of the plurality of mutant protein sequences, wherein the design score is based on Shannon entropy and log probability metrics calculated at each mutated position. The method further includes selecting, by the at least one processor, a subset of the plurality of mutant protein sequences having design scores that satisfy a predefined threshold. The method further includes predicting, for each mutant protein sequence in the selected subset, by the at least one processor, a corresponding three-dimensional structure. The method further includes computing, by the at least one processor, a stability score for each predicted structure based on one or more structural and sequence-based features. The method further includes outputting, by the at least one processor, a ranked list of thermostable mutant protein sequences based on the design score and the stability score.

In accordance with an embodiment, the method further includes performing, by the at least one processor, molecular dynamics simulations on the predicted structures under thermal stress conditions. The method further includes computing, by the at least one processor, dynamic simulation metrics comprising at least one of: RMSD variation, radius of gyration, solvent-accessible surface area, and hydrogen bond retention. The method further includes re-ranking, by at least one processor, the thermostable mutant protein sequences based on the dynamic simulation metrics.

In accordance with an embodiment, the solvent-exposed residues are identified using a neighbor search algorithm.

In accordance with an embodiment, the loop regions are inferred based on the spatial arrangement of atoms in the three-dimensional structure.

In accordance with an embodiment, the active site residues are determined based on proximity to a known ligand-binding region.

In accordance with an embodiment, the message-passing neural network is executed under multiple temperature parameters to simulate mutation generation at varying stringency levels.

In accordance with an embodiment, the molecular dynamics simulations on the predicted structures evaluate the compactness of the corresponding three-dimensional structure of each mutant protein sequence using radius of gyration.

In accordance with an embodiment, the molecular dynamics simulations on the predicted structures further evaluate local unfolding in the mutant structure over time.

In accordance with an embodiment, the fixed-position mask is dynamically generated based on evolutionary conservation scores derived from position-specific scoring matrices (PSSM).

In accordance with an embodiment, the at least one processor is further configured to prioritize mutations occurring in a hydrophobic core of the protein.

In accordance with an embodiment, the design score and stability score are combined using a machine learning model trained to identify high-stability protein variants. The machine learning model is the message-passing neural network.

In another aspect of the present disclosure, a system for generating thermostable variants of a protein is disclosed. The system includes a memory operatively associated with at least one processor, the memory including machine-executable instructions that, when executed by the at least one processor, cause the at least one processor to receive a three-dimensional structure of a target protein. A plurality of mutable regions is identified within the three-dimensional structure, and the plurality of mutable regions comprises solvent-exposed residues and loop regions. The at least one processor is further configured to perform a multiple sequence alignment of the target protein to identify a plurality of conserved residues in the target protein. The at least one processor is further configured to generate a fixed-position mask based on the plurality of conserved residues and a plurality of active site residues. The fixed-position mask defines a set of excluded residues that are not subject to mutation. The at least one processor is further configured to generate a plurality of mutant protein sequences using a message-passing neural network by introducing mutations at unmasked positions within the plurality of mutable regions. The at least one processor is further configured to compute a design score for each of the plurality of mutant protein sequences, the design score is based on Shannon entropy and log probability metrics calculated at each mutated position. The at least one processor is further configured to select a subset of the plurality of mutant protein sequences having design scores that satisfy a predefined threshold. The at least one processor is further configured to predict for each mutant protein sequence in the selected subset a corresponding three-dimensional structure. The at least one processor is further configured to compute a stability score for each predicted structure based on one or more structural and sequence-based features. The at least one processor is further configured to output a ranked list of thermostable mutant protein sequences based on the design score and the stability score.

In accordance with an embodiment, the at least one processor is further configured to: perform a molecular dynamics simulation for each predicted structure under thermal stress conditions; compute one or more dynamic simulation metrics for each predicted structure, wherein the dynamic simulation metrics comprise at least one of RMSD variation over time, radius of gyration, solvent-accessible surface area, and hydrogen bond retention and re-rank the selected mutant protein sequences based on the corresponding design score, the stability score, and the one or more dynamic simulation metrics.

In accordance with an embodiment, the solvent-exposed residues are identified using a neighbor search algorithm.

In accordance with an embodiment, the loop regions are inferred based on the spatial arrangement of atoms in the three-dimensional structure.

In accordance with an embodiment, the active site residues are determined based on proximity to a known ligand-binding region.

In accordance with an embodiment, the message-passing neural network is executed under multiple temperature parameters to simulate mutation generation at varying stringency levels.

In accordance with an embodiment, the temperature parameters include values selected from a group consisting of 0.1, 0.3, and 0.5.

In accordance with an embodiment, the design score is used to filter mutant protein sequences having a Shannon entropy less than 1.0 and a log probability greater than or equal to 0.5.

In accordance with an embodiment, the molecular dynamics simulations on the predicted structures evaluate the compactness of the corresponding three-dimensional structure of each mutant protein sequence using a radius of gyration.

It is to be understood that both the foregoing general description and the following detailed description are exemplary and explanatory only and are not restrictive of the invention, as claimed.

### BRIEF DESCRIPTION OF THE DRAWINGS

The above-mentioned implementations are further described herein with reference to the accompanying figures. It should be noted that the description and figures relate to exemplary implementations and should not be construed as a limitation to the present disclosure. It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalent thereof.

FIG. 1 is a diagram illustrating an exemplary environment of a system for generating thermostable variants of a protein, in accordance with an embodiment of the present disclosure.

FIG. 2 is a block diagram illustrating various modules of the system, in accordance with an embodiment of the present disclosure.

FIG. 3 illustrates an exemplary process flow for generating thermostable variants of the protein, in accordance with an embodiment of the present disclosure.

FIG. 4A illustrates an exemplary flow chart of a method for generating thermostable variants of the protein, in accordance with an embodiment of the present disclosure.

FIG. 4B illustrates an exemplary flow chart of a method for evaluating and re-ranking thermostable protein variants using molecular dynamics simulations, in accordance with an embodiment of the present disclosure.

FIG. 5 illustrates an exemplary environment suitable for implementing various embodiments of the present disclosure.

### DETAILED DESCRIPTION

In the following descriptions, certain specific details are set forth in order to provide a thorough understanding of various disclosed embodiments. However, one skilled in the relevant art will recognize that embodiments may be practiced without one or more of the specific details, or with other methods, components, materials, etc. In other instances, well-known structures of methods associated with the modular seating unit system have not been shown or described in detail to avoid unnecessarily obscuring descriptions of the embodiments.

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims.

In the specification, the term “comprising” shall be understood to have a broad meaning similar to the term “including” and will be understood to imply the inclusion of a stated integer or step or group of integers or steps but not the exclusion of any other integer or step or group of integers or steps. This definition also applies to variations on the term “comprising” such as “comprise” and “comprises.”

In the specification, the term “engage” and its variants including “engagement,” “engages,” “engaging,” and “engaged” as used herein are to be interpreted to include engagement by touching, rubbing, or abutting, including engagement in one or more of an axial, radial, tangential, and circumferential direction, and includes engagement through an intermediary such as a component positioned or sandwiched between the counter face and head of the fastener.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

Certain terminology used in the description is for convenience in reference only and shall not be limiting. For example, terms such as “secured environment,” “untrusted environment,” “sensitive data,” and “anonymized tokens” refer to the disclosed subject matter as described in the context of the invention. The words “inwardly,” “outwardly,” “forward,” and “backward” refer to directions toward and away from, respectively, the geometric center of the aspect being described and designated parts thereof. The terminology will include the words specifically mentioned, derivatives thereof, and words of similar meaning. Like reference numbers denote like features, components, or elements throughout the various embodiments.

In addition, as used herein, the term “or” is an inclusive “or” operator and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” includes plural references. The meaning of “in” includes “in” and “on.”

Features that are described in the context of an embodiment may correspondingly be applicable to the same or similar features in other embodiments. Features that are described in the context of an embodiment may correspondingly be applicable to other embodiments, even if not explicitly described in these other embodiments. Furthermore, additions and/or combinations and/or alternatives as described for a feature in the context of an embodiment may correspondingly be applicable to the same or similar feature in other embodiments.

Throughout the specification, the term “Three-Dimensional Structure” may refer to the atomic-level spatial configuration of a protein, typically represented as a Protein Data Bank (PDB) file or other structural formats. This structure captures the folding and positioning of amino acid residues and serves as the basis for identifying mutable regions, predicting mutant conformations, and conducting stability assessments.

Throughout the specification, the term “Mutable Regions” may refer to specific residues or segments within the protein structure that are eligible for mutation. These include, but are not limited to, solvent-exposed residues and loop regions, which are inferred based on spatial configuration and flexibility. Mutable regions are determined after excluding conserved residues and active site regions to preserve protein function.

Throughout the specification, the term “Fixed-Position Mask” may refer to a computational construct that identifies and excludes residues from mutation based on conservation analysis and proximity to functionally important regions such as the active site. The mask is applied during mutation generation to ensure that structural and functional integrity of the protein is retained.

Throughout the specification, the term “Message-Passing Neural Network” (MPNN) may refer to a type of graph-based deep learning model designed to generate protein sequence variants. The MPNN operates over the graph representation of the protein structure, generating mutation suggestions at unmasked positions while maintaining backbone compatibility.

Throughout the specification, the term “Design Score” may refer to a quantitative metric computed for each mutant sequence using outputs from the MPNN. The score is based on Shannon entropy and log-probability values associated with each mutated residue, and reflects the statistical confidence and likelihood of the sequence being naturally realizable.

Throughout the specification, the term “Stability Score” may refer to a computed measure of the structural integrity of a predicted mutant protein structure. The stability score may include one or more of: root mean square deviation (RMSD) from the wild-type, predicted Local Distance Difference Test (pLDDT) values, solvent-accessible surface area (SASA), sequence-based features, and embedding similarity derived from structure prediction models.

Throughout the specification, the term “Molecular Dynamics Simulation” (MD) may refer to a physics-based computational technique used to evaluate the dynamic behavior of a protein under thermal stress conditions. MD simulations are used to assess the flexibility, compactness, and structural stability of mutant proteins over time in an aqueous environment.

Throughout the specification, the term “Dynamic Simulation Metrics” may refer to the outputs derived from molecular dynamics simulations, including but not limited to: RMSD variation over time, radius of gyration (Rg), solvent-accessible surface area (SASA), and hydrogen bond retention. These metrics enable evaluation of protein behavior beyond static predictions.

Throughout the specification, the term “Thermostable Variant” may refer to a mutant protein sequence and structure that exhibits improved resistance to thermal denaturation while maintaining the original function of the wild-type protein. Thermostability is inferred through a combination of design scores, structural stability scores, and dynamic simulation metrics.

Proteins play an essential role in industrial and therapeutic applications, including pharmaceutical manufacturing and chemical processing. However, many naturally occurring proteins exhibit poor thermal stability, which limits their utility under industrial conditions involving elevated temperatures. The need to maintain protein functionality while improving thermostability has led to the exploration of computational protein engineering methods.

Conventional techniques for protein design, such as random mutagenesis or directed evolution, suffer from key limitations. These approaches often involve excessive experimental iterations and may disrupt protein activity by introducing mutations in conserved or functionally critical regions. While machine learning models and structure prediction tools have emerged in recent years, existing computational approaches are fragmented. They either focus exclusively on sequence-based prediction or overlook the impact of structural and dynamic behavior on protein function.

Some existing solutions rely on message-passing neural networks (MPNNs) to generate mutant protein sequences. However, these implementations generally apply mutations without restriction across the sequence, failing to preserve critical conserved regions and active sites. Additionally, the evaluation of these mutants is often based only on static structural metrics, without accounting for how the protein behaves under dynamic thermal stress, which is essential for understanding true thermostability.

To address these challenges, the present disclosure introduces a systematic and integrated approach to designing thermostable protein variants. The solution begins with identifying mutation-tolerant regions within the protein structure, specifically targeting solvent-exposed residues and loop regions, while masking conserved residues and active sites to preserve function. A message-passing neural network is then employed to generate mutations exclusively at permitted locations, with multiple runs at varying temperature parameters to capture consistent and high-confidence variants.

Mutants are scored using a combination of sequence and structure-based metrics, including Shannon entropy, log probability, RMSD, pLDDT, and solvent accessibility. Top-ranking candidates are further evaluated using classical molecular dynamics (MD) simulations under thermal stress conditions. This dynamic simulation layer enables assessment of long-term structural behavior, including RMSD variation over time, radius of gyration, hydrogen bond retention, and ligand-binding, ensuring that the functional and thermal properties of the protein are preserved or improved.

This multi-stage, constraint-driven pipeline reduces the dependency on trial-and-error methods, enables more accurate screening of thermostable variants, and ensures better functional integrity of the designed proteins. The framework is modular and may be extended to incorporate additional properties such as solubility, aggregation propensity, and pH stability with minimal architectural change.

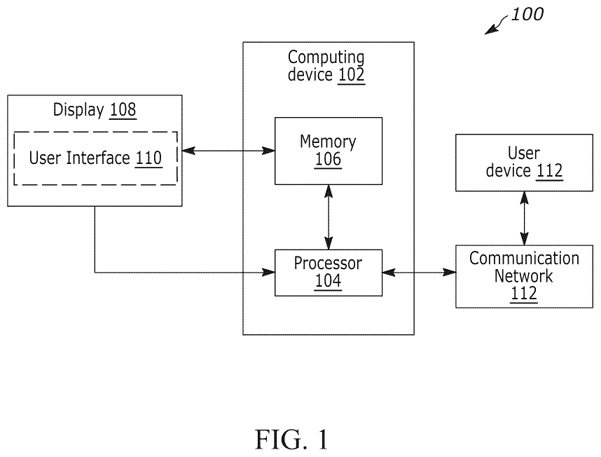

Hereafter, the invention is described in detail with reference to FIG. 1 to FIG. 5 as provided in the accompanying drawings.

Referring now to FIG. 1, an exemplary environment of a system 100 for generating thermostable variants of a protein is illustrated, in accordance with an embodiment of the present disclosure.

With reference to FIG. 1, the system 100 includes a computing device 102, a processor 104, a memory 106, a display 108, a user interface 110, a user device 112, and a communication network 114. In an embodiment, these components work together to generate thermostable variants of the protein. In an embodiment, the thermostable variant may be a mutant protein sequence and structure that exhibits improved resistance to thermal denaturation while maintaining the original function of the wild-type protein. Thermostability is inferred through a combination of design scores, structural stability scores, and dynamic simulation metrics.

The system 100 may implement the computing device 102 (for example, server, desktop, laptop, notebook, netbook, tablet, smartphone, mobile phone, or any other computing device). The computing device 102 may be configured to verify sensitive personal information in the decentralized ecosystem. Examples of sensitive personal information may include personally identifiable information (PII) such as a user's full name, date of birth, social security number, national identification number, passport number, driver's license number, and residential address.

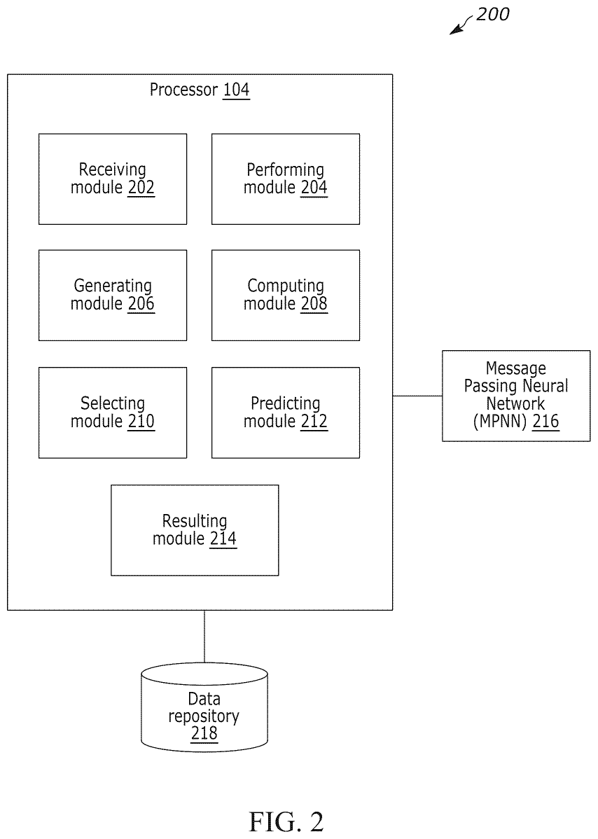

As will be described in greater detail in conjunction with FIGS. 2-5, in order to generate thermostable variants of the protein, the computing device 102 first receives a three-dimensional structure of a target protein. A plurality of mutable regions is identified within the three-dimensional structure, and the plurality of mutable regions comprises solvent-exposed residues and loop regions. Further, the computing device 102 is configured to perform a multiple sequence alignment of the target protein to identify a plurality of conserved residues in the target protein. Further, the computing device 102 is configured to generate a fixed-position mask based on the plurality of conserved residues and a plurality of active site residues. The fixed-position mask defines a set of excluded residues that are not subject to mutation. Further, the computing device 102 is configured to generate a plurality of mutant protein sequences using a message-passing neural network by introducing mutations at unmasked positions within the plurality of mutable regions. Further, the computing device 102 is configured to compute a design score for each of the plurality of mutant protein sequences, the design score is based on Shannon entropy and log probability metrics calculated at each mutated position. Further, the computing device 102 is configured to select a subset of the plurality of mutant protein sequences having design scores that satisfy a predefined threshold. Further, the computing device 102 is configured to predict for each mutant protein sequence in the selected subset a corresponding three-dimensional structure. Further, the computing device 102 is configured to compute a stability score for each predicted structure based on one or more structural and sequence-based features. Further, the computing device 102 is configured to output a ranked list of thermostable mutant protein sequences based on the design score and the stability score.

The communication network 114 serves as the medium facilitating data transfer between the computing device 102 and the user device 112. The communication network 114 may be wired, wireless, or any combination of wired and wireless communication networks, such as cellular networks, Wi-Fi, the internet, local area networks (LANs), or the like. In one embodiment, the communication network 114 may include one or more networks such as a data network, a wireless network, a telephony network, or any combination thereof. The data network may be any local area network (LAN), metropolitan area network (MAN), wide area network (WAN), a public data network (e.g., the Internet), short-range wireless network, or any other suitable packet-switched network, such as a commercially owned, proprietary packet-switched network, e.g., a proprietary cable or fiber-optic network, and the like, or any combination thereof. In addition, the wireless network may be, for example, a cellular network and may employ various technologies including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., worldwide interoperability for microwave access (WiMAX), Long Term Evolution (LTE) networks, code division multiple access (CDMA), wideband code division multiple access (WCDMA), wireless fidelity (Wi-Fi), wireless LAN (WLAN), Bluetooth®, Internet Protocol (IP) data casting, satellite, mobile ad-hoc network (MANET), and the like, or any combination thereof.

The user device 112 may include a range of devices, such as a smartphone, tablet, desktop computer, or laptop. These devices represent various types of user equipment that can access and interact with the Computing device 102 via the communication network 114. Each device may be configured to send and receive data across the network in a manner that ensures secure and reliable interaction with the system 100. A person of ordinary skill in the art will appreciate that the user device 112 may include, but is not limited to intelligent, multi-sensing, network-connected devices, which may integrate seamlessly with each other and/or with a central server or a cloud-computing system or any other device that is network-connected.

Additionally, in some embodiments, the user device 112 may include, but is not limited to, a handheld wireless communication device (e.g., a mobile phone, a smartphone, a phablet device, and so on), a wearable computer device (e.g., a head-mounted display computer device, a head-mounted camera device, a wristwatch computer device, and so on), a Global Positioning System (GPS) device, a laptop computer, a tablet computer, or another type of portable computer, a media playing device, a portable gaming system, and/or any other type of computer device with wireless communication capabilities, and the like. In an embodiment, the user device 112 may include, but is not limited to, any electrical, electronic, electromechanical, or equipment, or a combination of one or more of the above devices, such as virtual reality (VR) devices, augmented reality (AR) devices, laptop, a general-purpose computer, desktop, personal digital assistant, tablet computer, mainframe computer, or any other computing device. The user device 112 may include one or more in-built or externally coupled accessories including, but not limited to, a visual aid device such as a camera, an audio aid, a microphone, a keyboard, and input devices for receiving input from the user or the entity such as a touchpad, touch-enabled screen, electronic pen, and the like. A person of ordinary skill in the art will appreciate that the user device 112 may not be restricted to the mentioned devices and various other devices may be used.

The computing device 102 is communicatively coupled to the at least one processor (processor 104) and the memory 106, which together form the core processing unit of the system 100. The processor 104 is responsible for executing instructions stored in the memory 106, which include various modules for receiving, performing, generating, computing, selecting, predicting, and outputting steps of the present disclosure. Each module is explained in detail in conjunction with FIG. 2. The memory 106 may include any non-transitory storage device including, for example, volatile memory such as random-access memory (RAM), or non-volatile memory such as erasable programmable read only memory (EPROM), flash memory, and the like. Further, the memory 106 stores processor-executable instructions, which, when executed by the processor 104, enable the system 100 to generate thermostable variants of the protein. The display 108 and the user interface 110 serve as the primary means of interaction between the user and the system 100. The user interface 110 captures user inputs, user inputs, such as the selection of target proteins, adjustment of design parameters (e.g., temperature thresholds, masking constraints), or upload of structural files. It also presents outputs, including ranked lists of mutant protein sequences, visual representations of predicted structures, and associated scoring metrics such as entropy, RMSD, or predicted stability scores, via the display 108.

The communication network 114 enables real-time communication between the user device 112 and the computing device 102, facilitating the execution of resource-intensive processes such as structure prediction, sequence generation using MPNN, and molecular dynamics simulations. In some embodiments, the communication network 114 may represent various network types, including, but not limited to, cloud-based computing environments, high-performance computing clusters, or local computational infrastructure. The communication network 114 ensures that all data transactions between user inputs, computational services, and output delivery are handled securely, efficiently, and in compliance with research data integrity protocols.

FIG. 2 is a block diagram 200 illustrating various modules of the system 100, in accordance with an embodiment of the present disclosure. FIG. 2 is explained in conjunction with elements of FIG. 1. The block diagram 200 includes the memory 106, which is configured to store various modules to generate thermostable variants of the protein. The modules include a receiving module 202, a performing module 204, a generating module 206, a computing module 208, a selecting module 210, a predicting module 212, and a resulting module 214. Each module performs specific functions to ensure that the thermostable variants of the protein are efficiently generated.

In an embodiment, the receiving module 202 is configured to receive a three-dimensional structure of a target protein. In an embodiment, the received structure may be sourced from experimental data, such as X-ray crystallography or NMR studies, or predicted using structure prediction tools. Upon receipt, the system initiates structural preprocessing to resolve any missing atoms or irregular residues to ensure the protein model is suitable for computational analysis. A plurality of mutable regions is then identified within the structure; these include solvent-exposed residues and loop regions, which are considered tolerant to mutations. The solvent-exposed residues are identified using a neighbor search algorithm, which evaluates spatial proximity between residues and solvent molecules, indicating surface accessibility. The loop regions are inferred based on the spatial arrangement of atoms, typically lacking defined secondary structure, and representing flexible regions ideal for mutational analysis.

The performing module 204 is configured to perform a multiple sequence alignment (MSA) of the target protein to identify a plurality of conserved residues in the target protein. The MSA involves aligning the protein sequence against homologous sequences retrieved from biological databases to identify conserved regions. These conserved residues are typically critical for maintaining structural fold and biological activity, and thus are flagged to be preserved during mutation. The MSA results serve as a foundational input to the generating module 206 by guiding which residues should be masked and protected from alteration. This ensures that mutations introduced later in the pipeline do not disrupt functionally essential or evolutionarily preserved regions of the protein

Further, the generating module 206 is configured to generate a fixed-position mask based on the plurality of conserved residues and a plurality of active site residues. The fixed-position mask defines a set of excluded residues that are not subject to mutation. The active site residues are determined based on proximity to a known ligand-binding region, ensuring the catalytic or binding functions of the protein are not disturbed. Once generated, the mask is applied during mutation generation to prevent undesired changes in critical areas, thereby improving the likelihood that the engineered protein retains its original activity. This masking mechanism forms a core part of the intelligent constraint strategy embedded in the design framework.

Further, the generating module 206 is configured to generate a plurality of mutant protein sequences using a message-passing neural network (MPNN) 216. The MPNN 216 receives the fixed-position mask and protein structure as input and introduces mutations only at unmasked, mutation-permissible positions. This ensures that the proposed mutations are structurally feasible and do not affect conserved or functionally important regions. In an embodiment, the MPNN 216 is executed under multiple temperature parameters, which simulate mutation generation at varying stringency levels. These temperature parameters (e.g., 0.1, 0.3, and 0.5) influence the diversity and confidence of the generated sequences, with higher values allowing more exploration of sequence space. By running the MPNN 216 at multiple temperatures and aggregating results, the system identifies mutations that are robust, consistent, and statistically significant across different design scenarios.

Further, the computing module 208 is configured to compute a design score for each of the plurality of mutant protein sequences. The design score is based on Shannon entropy and log probability metrics calculated at each mutated position. In an embodiment, Shannon entropy is computed to measure the variability or uncertainty of amino acid substitutions across multiple runs of the neural network under different temperature parameters. For a given residue position, a low entropy value indicates that the same amino acid substitution consistently appears across different model configurations, signaling high model confidence and biological plausibility. Conversely, high entropy reflects a lack of consensus and implies uncertainty, making such mutations less desirable for further validation. The log probability reflects the model's internal belief about the likelihood of the mutated residue at a given position, conditioned on the structural context and surrounding sequence. A higher log probability indicates that the substitution is statistically favoured by the trained model and likely to exist in naturally occurring homologs. In an embodiment, the design score is used as a filtering mechanism to eliminate low-confidence or improbable sequences. Specifically, sequences with entropy values less than 1.0 and log probability values greater than or equal to 0.5 are retained for further evaluation. This scoring step ensures that only high-confidence, biologically meaningful variants are carried forward in the pipeline, thereby increasing the reliability and efficiency of the protein design process.

Further, the selecting module 210 is configured to select a subset of the plurality of mutant protein sequences having design scores that satisfy a predefined threshold. For example, the predefined threshold may include a Shannon entropy less than 1.0 and a log probability greater than or equal to 0.5. These thresholds help ensure that only high-confidence and statistically plausible mutations are carried forward for further structural and stability evaluation. This selection step significantly reduces the number of candidate variants by discarding sequences that are unlikely to exhibit favorable stability or structural compatibility. The selected subset represents the most promising mutations for structure prediction and thermodynamic evaluation. This process minimizes computational overhead and ensures that resources are focused only on high-potential candidates, enhancing the overall throughput of the protein engineering workflow.

Further, the predicting module 212 is configured to predict for each mutant protein sequence in the selected subset a corresponding three-dimensional structure. This is typically performed using advanced structure prediction tools such as ESM Fold or other neural network-based protein structure predictors. These tools take the mutated sequence and estimate the folding behavior and spatial arrangement of the residues in three-dimensional space. The resulting mutant structures are then compared against the wild-type structure to assess whether the introduced mutations have significantly altered the global or local conformation of the protein. This structural modeling step is critical to ensure that the designed variants are structurally viable and functionally foldable.

Further, the computing module 208 is configured to compute a stability score for each predicted structure based on one or more structural and sequence-based features such as RMSD, solvent-accessible surface area (SASA), predicted local distance difference test (pLDDT) scores, and embedding similarity. These metrics collectively evaluate how stable the mutant protein is likely to be under normal or elevated temperatures. In one embodiment, the stability score is combined with the design score using a machine learning model, specifically, the same MPNN model, to generate a composite score that reflects both mutation confidence and structural robustness. This dual-scoring framework enhances prediction accuracy and prioritizes variants that are both biophysically stable and evolutionarily plausible.

Further, the resulting module 214 is configured to output a ranked list of thermostable mutant protein sequences. This ranking is derived from a combination of design scores and stability scores, allowing for a general assessment of each variant's potential effectiveness. In one embodiment, the performing module 204 is further configured to prioritize mutations occurring in the hydrophobic core of the protein, as these often contribute significantly to thermostability. The final ranked list may be visualized, stored, or exported for molecular dynamics simulation or for experimental validation in the lab. This ranking serves as a data-driven, interpretable, and high-confidence recommendation for selecting thermostable protein variants suitable for industrial or therapeutic use.

In some embodiments, the performing module 204 performs a molecular dynamics simulation for each predicted structure under thermal stress conditions. In an embodiment, the molecular dynamics simulations on the predicted structures evaluate the compactness of the corresponding three-dimensional structure of each mutant protein sequence using radius of gyration. Further, the computing module 208 computes one or more dynamic simulation metrics for each predicted structure, the dynamic simulation metrics include at least one of RMSD variation over time, radius of gyration, solvent-accessible surface area, and hydrogen bond retention. The resulting module 214 re-ranks the selected mutant protein sequences based on the corresponding design score, the stability score, and the one or more dynamic simulation metrics.

In an embodiment, the system 100 utilizes the MPNN, a machine learning-based protein sequence design tool, to optimize the thermostability of a target protein. By using the structural information of the protein, MPNN may generate sequence variants predicted to exhibit increased thermostability. These variants may then be evaluated computationally using thermostability prediction tool like TemStaPro (Temperatures of Stability for Proteins) or any equivalent tool. ESM fold may be used to generate the model of the structure of the protein. The mutate sequences may be evaluated using TemStaPro and ddG. In case, the mutated variant has a score above the threshold, it may be analyzed by Replica-exchange MD at desired temperatures.

In an embodiment, the data repository 218 is configured to store and manage all data artifacts generated and utilized during the thermostable protein variant design process. This includes, but is not limited to, the original three-dimensional structures of target proteins, multiple sequence alignment results, conserved residue mappings, mutation masks, generated mutant sequences, predicted structures, and associated scoring metrics such as design scores, stability scores, and simulation-derived data.

The data repository 218 may also store historical mutation data, temperature-based MPNN outputs, and dynamic simulation metrics such as RMSD trajectories, radius of gyration plots, and ddG binding affinity scores. This comprehensive storage enables traceability, version control, and reproducibility of design workflows.

In some embodiments, the data repository 218 supports structured queries and indexing, allowing researchers or downstream applications to retrieve and analyze specific variants, scoring thresholds, or protein-specific mutation patterns. It may be implemented using a relational database, NoSQL system, or object store, depending on the scale and access requirements of the deployment environment.

As will be appreciated, all such aforementioned modules 202-218 may be represented as a single model or a combination of different models. Further, as will be appreciated by those skilled in the art, each of the modules 202-218 may reside, in whole or in parts, on one device or multiple devices in communication with each other. In some embodiments, each of the modules 202-218 may be implemented as dedicated hardware circuit comprising custom application-specific integrated circuit (ASIC) or gate arrays, off-the-shelf semiconductors such as logic chips, transistors, or other discrete components. Each of the modules 202-218 may also be implemented in a programmable hardware device such as a field programmable gate array (FPGA), programmable array logic, programmable logic device, and so forth. Alternatively, each of the modules 202-218 may be implemented in software for execution by various types of processors (e.g., processor 104). An identified module of executable code may, for instance, include one or more physical or logical blocks of computer instructions, which may, for instance, be organized as an object, procedure, function, or other construct. Nevertheless, the executables of the modules 202-218 need not be physically located together but may include disparate instructions stored in different locations which, when joined logically together, include the model and achieve the stated purpose of the model. Indeed, a module of executable code could be a single instruction, or many instructions, and may even be distributed over several different code segments, among different applications, and across several memory devices.

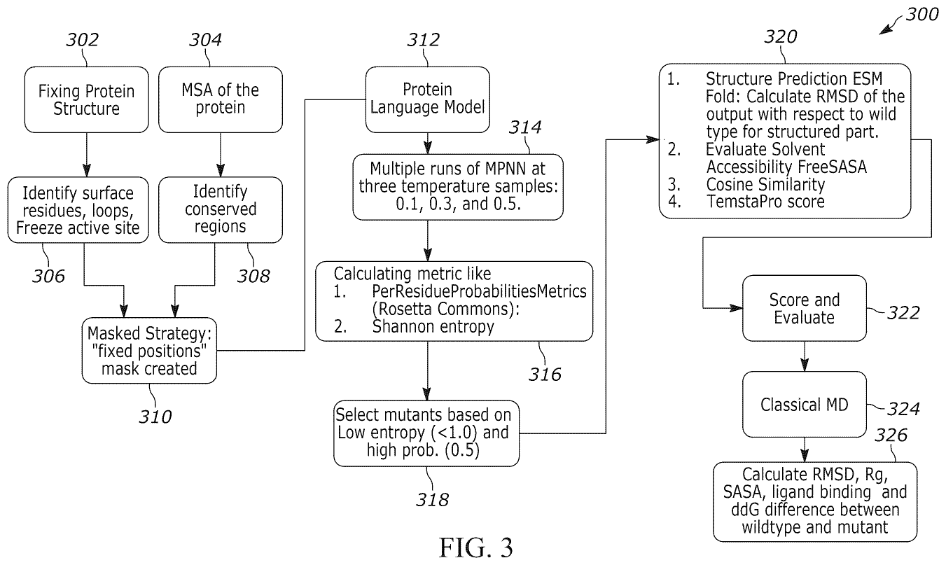

Referring now to FIG. 3, an exemplary process flow 300 for generating thermostable variants of the protein is illustrated, in accordance with an embodiment of the present disclosure. It will be understood that each block of the flow diagram of the method 500 may be implemented by various means, such as hardware, firmware, processors, circuitry, and/or other communication devices associated with executing software, including one or more computer program instructions. For example, the procedures described in this method may be embodied by computer program instructions. In this regard, the computer program instructions that embody the procedures described above may be stored in a memory of a computing device, employing an embodiment of the present disclosure, and executed by a processor.

These computer program instructions may be stored in a computer-readable memory, which may direct a computer or other programmable apparatus to function in a specified manner. By doing so, the instructions stored in the computer-readable memory produce an article of manufacture, the execution of which implements the function specified in the flowchart blocks. The instructions may also be loaded onto a computer or programmable apparatus to initiate a series of operations on the device, resulting in a computer-implemented process in which the instructions executing on the apparatus provide operations that implement the functions specified in the flow diagram blocks.

Accordingly, the blocks of the flow diagram support combinations of means for performing the specified functions and combinations of operations for performing these functions. It will also be understood that one or more blocks of the flow diagram, and combinations of blocks within it, may be implemented by special-purpose hardware-based computer systems or a combination of special-purpose hardware and computer instructions. FIG. 3 is explained in conjunction with elements of FIG. 1 and FIG. 2. The method 300 may be implemented by various modules of the computing device 102.

In order to generate thermostable variants of the protein, at step 302, the process flow 300 begins by receiving a three-dimensional (3D) structure of a target protein. This structure may be derived from experimental sources (e.g., crystallography or NMR) or computational predictions. In many instances, the raw structure may have missing atoms or irregularities, particularly in flexible loop regions. To ensure structural consistency, a preprocessing step is carried out using structure correction tools such as PDBFixer, which identifies and fills missing atoms or residues. This ensures that the subsequent analysis and mutation modeling are performed on a clean, chemically valid structure that accurately represents the protein's native conformation.

At step 304, the processor 104 further performs a Multiple Sequence Alignment (MSA) of the target protein against a database of homologous sequences. The objective of this step is to identify evolutionarily conserved residues, those amino acid positions that remain unchanged across different species or variants of the same protein family. Conserved residues typically play critical roles in protein folding, structural integrity, and function. By mapping conservation information onto the protein structure, the system gains insight into which regions should remain unaltered to avoid disrupting essential biological properties.

At step 306, based on the three-dimensional structure, the process 300 further identifies solvent-exposed residues, loop regions, and active site residues. Solvent-exposed residues are those located on the surface of the protein and are more amenable to mutation without disturbing the protein's core stability. These are typically detected using a neighbor search algorithm or solvent accessibility calculations. Loop regions, often associated with flexibility, are inferred based on the spatial arrangement and secondary structure annotations in the protein. Conversely, the active site, where substrate binding or catalysis occurs, is frozen and excluded from mutation, as any change in this region could compromise the protein's biological function. The combination of these structural features helps the system define a targeted set of regions for potential mutation.

At step 308, the processor 104 identifies conserved regions of the protein from the MSA results, which are also excluded from mutation. These residues are also marked as non-mutable, given their essential role in maintaining the protein's functional fold. By integrating information from both the sequence alignment and structural analysis, the system ensures that only mutation-tolerant regions, such as non-conserved, surface-exposed, and loop regions, are considered for variation. This multi-layered protection mechanism is key to preserving function while modifying stability.

At step 310, a fixed-position mask is created. This mask defines the set of positions that are allowed or disallowed for mutation during sequence generation. This is a binary or index-based representation that labels each residue in the protein as either mutable or immutable. The mask is created by combining data from the structural analysis (step 306) and conservation analysis (step 308). Residues in conserved or active-site regions are marked as excluded, while all others in permissible zones (e.g., surface loops) are marked as candidate positions for mutation. This fixed-position mask is later used to guide the mutation generation algorithm, ensuring that changes are introduced only where safe and potentially beneficial.

At step 312, the processor 104 employs a protein language model, specifically a Message Passing Neural Network (MPNN), to generate mutant sequences at the unmasked positions. The MPNN is a deep learning model trained to operate on protein graphs, where nodes represent residues and edges represent spatial or sequential relationships. In this step, the MPNN receives the original protein structure and the fixed-position mask and generates mutant sequences by proposing substitutions only at the allowed (unmasked) positions. This targeted design approach ensures that the generated mutants retain structural plausibility while exploring the space of possible thermostabilizing changes.

At step 314, the MPNN is executed in multiple runs, each with a different temperature sampling parameter (e.g., 0.1, 0.3, and 0.5), to explore a broader mutational landscape and assess mutation recurrence. These temperature parameters influence the stringency and diversity of the output sequences. Lower values bias the model toward high-confidence, conservative mutations, while higher values allow greater variation. By aggregating outputs across multiple runs, the system identifies consistently recurring mutations, which are more likely to be both structurally viable and functionally neutral. This adds robustness and statistical confidence to the mutation selection process

At step 316, metrics such as PerResidueProbabilitiesMetric (e.g., from Rosetta Commons) and Shannon entropy are calculated for each position to quantify the confidence and diversity of the mutations. PerResidueProbabilitiesMetric, which evaluates how likely each residue in the mutant sequence is according to the MPNN model, based on learned biological priors. Shannon entropy, computed across different MPNN runs, to measure uncertainty and variability at each mutated position. Low entropy suggests stable, repeatable mutation choices, whereas high entropy indicates uncertainty. These metrics help in the quantitative scoring of mutant sequences and guide the selection of high-confidence candidates for further structural and functional evaluation.

At step 318, mutants with low entropy (<1.0) and high probability (≥0.5) are selected as high-confidence candidates for further evaluation. Only the variants satisfying both thresholds are retained, significantly reducing the pool of candidates and focusing analysis on the most promising mutant sequences.

At step 320, the structure of each selected mutant is predicted using tools such as ESM Fold, and the system evaluates:
Root Mean Square Deviation (RMSD) with respect to the wild-type structure,
Solvent Accessibility using FreeSASA,.
Cosine Similarity between embedding vectors,
Thermostability scores using a tool such as TemStaPro.

Once the structures are predicted, the processor 104 evaluates their similarity to the wild-type structure using RMSD for structural displacement, FreeSASA for solvent accessibility, cosine similarity between embedding vectors to assess overall fold similarity, and TemStaPro score to estimate thermostability. This step provides a comprehensive structural characterization of each mutant.

At step 322, the processor 104 performs a scoring and evaluation phase where the mutant structures are assessed and ranked based on the combined structural and sequence-based metrics. This multi-factorial evaluation ensures that only those variants that are structurally sound and thermodynamically favorable are advanced to the next phase.

At step 324, the top-ranking candidates are subjected to Classical Molecular Dynamics (MD) simulations to assess their behavior under thermal stress conditions. Unlike static models, MD captures temporal changes, enabling the observation of unfolding events, domain flexibility, or local destabilizations that may not be apparent from structure prediction alone. This provides critical insights into the real-world stability of the designed variants.

At step 326, the processor 104 calculates dynamic simulation metrics, including RMSD over time to monitor global stability, Radius of Gyration (Rg) to assess compactness, Solvent-Accessible Surface Area (SASA) to evaluate exposure and folding, ligand binding stability, and ddG differences between wild-type and mutant structures to assess ligand binding affinity and functional integrity compared to the wild-type. These metrics are used to further refine and confirm the thermostability and functional preservation of the designed variants. By analyzing these metrics, the system performs a final validation to identify mutants that not only show computationally predicted stability but also demonstrate robust dynamic behavior under thermal stress.

For a better understanding of the process 300, consider a use case where a wild-type enzyme commonly used in pharmaceutical manufacturing, such as lipase, needs to be engineered for improved thermostability without losing its catalytic function.

The process begins with the user submitting the three-dimensional (3D) structure of the wild-type lipase protein to the system. The submitted structure may be an experimental structure from the Protein Data Bank (PDB) or a predicted model. At this point, the system initiates structure preprocessing to identify and correct any missing atoms, loops, or irregular coordinates, ensuring that the input structure is suitable for computational manipulation.

Following structure correction, the system performs a Multiple Sequence Alignment (MSA) using known homologs of the lipase enzyme to identify evolutionarily conserved residues. These conserved residues often contribute to the protein's core fold and functional sites and must therefore be protected from mutagenesis.

In parallel, the system analyzes the protein structure to identify surface-exposed residues and flexible loop regions, using solvent accessibility metrics and spatial geometry. These regions are more tolerant to mutation and form the primary candidates for thermostability engineering. The active site, determined based on the enzyme's known ligand-binding region, is also identified and frozen, i.e., excluded from mutation to preserve catalytic activity.

With this information, the system generates a fixed-position mask, labeling each residue in the protein as either mutable or immutable. Conserved and active-site residues are marked as immutable, while surface-exposed and non-conserved residues are marked as eligible for mutation.

Next, the system utilizes the MPNN that acts as a protein language model. This model receives the protein structure and the fixed-position mask and generates mutant sequences by proposing substitutions only at the allowed positions. To improve mutation robustness, the system performs multiple MPNN runs, each configured with different temperature parameters (e.g., 0.1, 0.3, and 0.5). These temperature values modulate the randomness of sequence generation, simulating mutation stringency. Variants that appear consistently across multiple runs are considered more reliable.

For each mutation candidate, the system computes a design score, using metrics such as Shannon entropy and log-probability from the MPNN model. Low entropy and high probability indicate mutations that the model considers stable and biologically plausible. For example, a mutation where leucine is substituted by valine on the protein surface may be assigned a high probability with low entropy, suggesting that such a mutation is consistent with natural sequence variation.

Once filtered, the selected mutant sequences are processed for structure prediction using ESM Fold. For each predicted mutant structure, the system calculates:
RMSD to evaluate how much the mutant deviates from the wild-type structure,
Solvent Accessible Surface Area (SASA) to assess surface folding,
Cosine similarity between structure embeddings to assess conformational similarity,
And TemStaPro scores to predict thermostability across temperature gradients.

These metrics are combined to rank the mutant proteins. For instance, a variant that differs minimally from the wild-type in 3D structure but scores higher in predicted thermostability will be ranked higher.

The top-ranking variants, say, 5 or 10 candidates, are subjected to Classical Molecular Dynamics (MD) simulations. For the lipase variant, the system simulates how the protein behaves in a water box at elevated temperatures (e.g., 60° C.) for several nanoseconds. During the simulation, the system records:
How the RMSD evolves over time,
Whether the hydrophobic core remains intact,
How compact the structure is via the radius of gyration (Rg),
And how many hydrogen bonds are retained.
Additionally, the system checks the ligand binding ability of the mutant using ΔΔG calculations to ensure that catalytic function is not compromised.

At the end of this pipeline, the system presents a final list of thermostable mutant sequences, ranked based on structural preservation, dynamic stability, and activity retention. For instance, the system may identify a lipase mutant with substitutions at three surface-exposed residues that improves RMSD stability under thermal stress by 20% and shows no loss in ligand-binding energy. This variant is then selected for experimental validation.

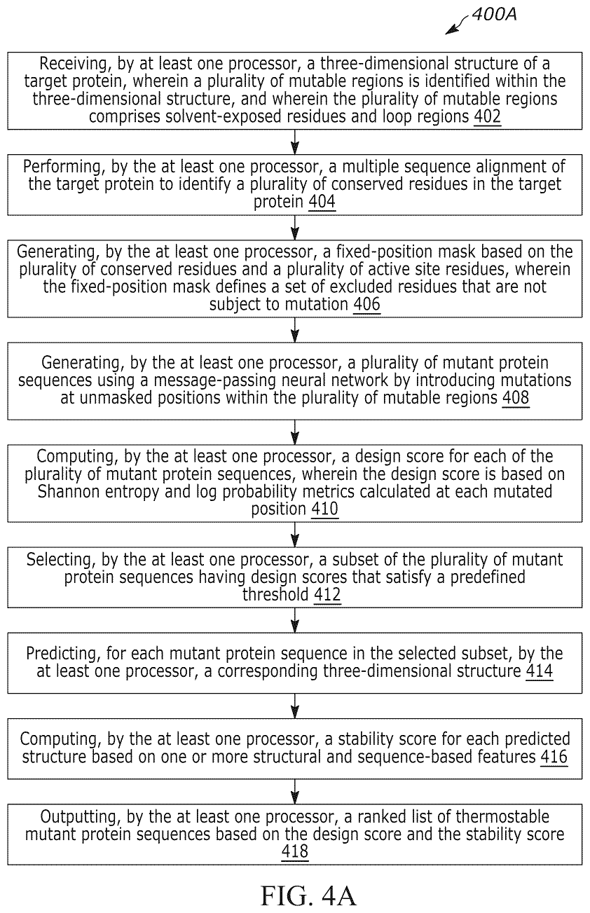

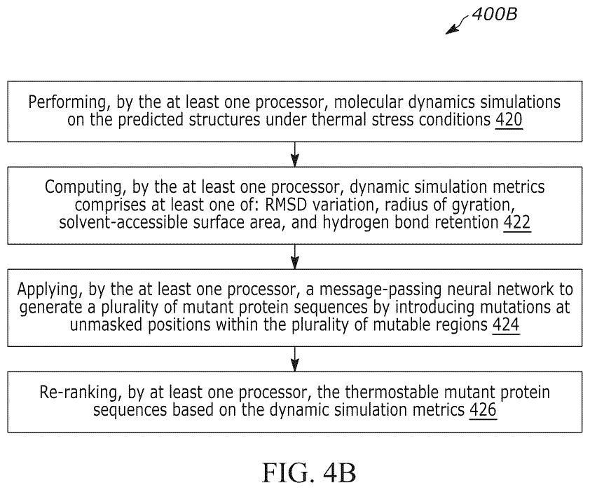

Referring now to FIG. 4A, an exemplary flow chart of a method 400A for generating thermostable variants of the protein is illustrated, in accordance with an embodiment of the present disclosure. FIG. 4A is explained in conjunction with elements of FIG. 1, FIG. 2, and FIG. 3. The method 400A may be implemented by at least one processor 104 of the system 100.

At step 402, method 400A includes receiving, by at least one processor, a three-dimensional structure of a target protein. Upon receiving the structure, the processor identifies a plurality of mutable regions within it. These mutable regions include solvent-exposed residues and loop regions, which are considered tolerant to mutation due to their surface accessibility and structural flexibility. The solvent-exposed residues are identified using a neighbor search algorithm, which evaluates spatial proximity to solvent molecules or other residues. The loop regions are inferred based on the spatial arrangement of atoms and lack of defined secondary structure (e.g., α-helix or β-sheet). By focusing on these regions, the system avoids altering structurally or functionally critical areas, such as the hydrophobic core or catalytic residues.

At step 404, the method 400A further includes performing, by the at least one processor, a multiple sequence alignment (MSA) of the target protein to identify a plurality of conserved residues in the target protein. In particular, the MSA compares the sequence of the input protein with known homologs to identify evolutionarily conserved residues, which are typically essential for folding or function. These conserved residues serve as constraints in the mutation process.

At step 406, the method 400A further includes generating, by the at least one processor, a fixed-position mask based on the plurality of conserved residues and a plurality of active site residues. The fixed-position mask defines a set of excluded residues that are not subject to mutation. The active site residues may be determined by evaluating the spatial proximity to known ligand-binding regions within the 3D structure. In one embodiment, the fixed-position mask is dynamically constructed using evolutionary conservation scores derived from Position-Specific Scoring Matrices (PSSMs), which quantitatively represent how conserved each amino acid is across related sequences. This mask guides the mutation generation process, ensuring only non-critical positions are modified.

At step 408, the method 400A further includes generating, by the at least one processor, a plurality of mutant protein sequences using a message-passing neural network by introducing mutations at unmasked positions within the plurality of mutable regions. The MPNN operates on a graph-based representation of the protein and is trained to suggest mutations that are structurally feasible and thermodynamically favorable. Multiple runs of the MPNN may be performed to capture mutation consistency and generate a diverse set of plausible variants. In an embodiment, the message-passing neural network is executed under multiple temperature parameters to simulate mutation generation at varying stringency levels.

At step 410, the method 400A further includes computing, by the at least one processor, a design score for each of the plurality of mutant protein sequences. The design score is based on Shannon entropy and log probability metrics calculated at each mutated position. The Shannon entropy reflects the confidence and stability of the mutation across different MPNN runs. The log probability measures the likelihood of the mutation being biologically plausible according to the model.

At step 412, the method 400A further includes selecting, by the at least one processor, a subset of the plurality of mutant protein sequences having design scores that satisfy a predefined threshold. For example, low entropy (<1.0) and high log probability (≥0.5) are selected for further structural evaluation. This step ensures that only high-confidence, biologically meaningful variants are carried forward in the design pipeline.

At step 414, the method 400A further includes predicting, for each mutant protein sequence in the selected subset, by the at least one processor, a corresponding three-dimensional structure. In an embodiment, the sequence is predicted using structure prediction tools, such as ESM Fold or similar models. These predicted structures provide a basis for evaluating how the mutations have affected overall protein folding, packing, and surface properties. The predicted structures are compared against the wild-type reference to ensure that the variants have not undergone unfavorable conformational changes.

At step 416, the method 400A further includes computing, by the at least one processor, a stability score for each predicted structure This score is based on one or more structural and sequence-based features, including but not limited to, root mean square deviation (RMSD), solvent-accessible surface area (SASA), pLDDT scores, cosine similarity, and physicochemical properties derived from sequence embeddings. In one embodiment, the design score and stability score are further combined using a machine learning model, such as the same message-passing neural network, which is trained to predict the overall stability of protein variants based on multi-modal input. This enhances predictive accuracy and reduces false positives in thermostability prediction.

At step 418, the method 400A further includes outputting, by the at least one processor, a ranked list of thermostable mutant protein sequences based on the design score and the stability score. In an embodiment, the at least one processor is configured to prioritize mutations that occur in or near the hydrophobic core, as stabilizing this region may significantly improve protein thermostability. The final ranked list serves as the output for the static evaluation phase and may be further subjected to dynamic simulation (as will be described in FIG. 4B) for final validation and refinement.

Referring now to FIG. 4B, an exemplary flow chart of a method 400B for evaluating and re-ranking thermostable protein variants using molecular dynamics simulations is illustrated, in accordance with an embodiment of the present disclosure. FIG. 4B is explained in conjunction with elements of FIG. 1, FIG. 2, FIG. 3, and FIG. 4A. The method 400B may be implemented by at least one processor 104 of the system 100.

Once the output list of thermostable mutant protein sequences is generated using the design and stability scoring methods described in FIG. 4A, the method 400B proceeds to perform a secondary validation based on molecular dynamics (MD) simulations. At step 420 perform, by the at least one processor, molecular dynamics simulations on the predicted structures under thermal stress conditions. In an embodiment, the molecular dynamics simulations on the predicted structures evaluate the compactness of the corresponding three-dimensional structure of each mutant protein sequence using radius of gyration. In an embodiment, the molecular dynamics simulations on the predicted structures further evaluate local unfolding in the mutant structure over time. These simulations model the time-dependent behavior of the proteins in a solvated environment at elevated temperatures. In one embodiment, the simulations evaluate the compactness of each three-dimensional mutant structure using radius of gyration (Rg) as a metric. In another embodiment, the simulations further assess local unfolding within the protein, capturing dynamic instabilities or segmental motion that could compromise thermostability.

At step 422, the method 400B further includes computing, by the at least one processor, dynamic simulation metrics. These include, but are not limited to, RMSD variation, radius of gyration, solvent-accessible surface area, and hydrogen bond retention. These metrics provide a more realistic and temporal view of the protein's stability than static models.

At step 424, the method 400B a message-passing neural network is used to generate a plurality of mutant protein sequences by introducing mutations at unmasked positions within the plurality of mutable regions.

At step 426, the method 400B further includes re-ranking, by at least one processor, the thermostable mutant protein sequences based on the dynamic simulation metrics. This re-ranking process integrates the time-resolved behavior of each variant to refine the overall assessment of thermostability, ensuring that only those variants demonstrating both static structural integrity and dynamic resilience are prioritized for further validation or deployment.

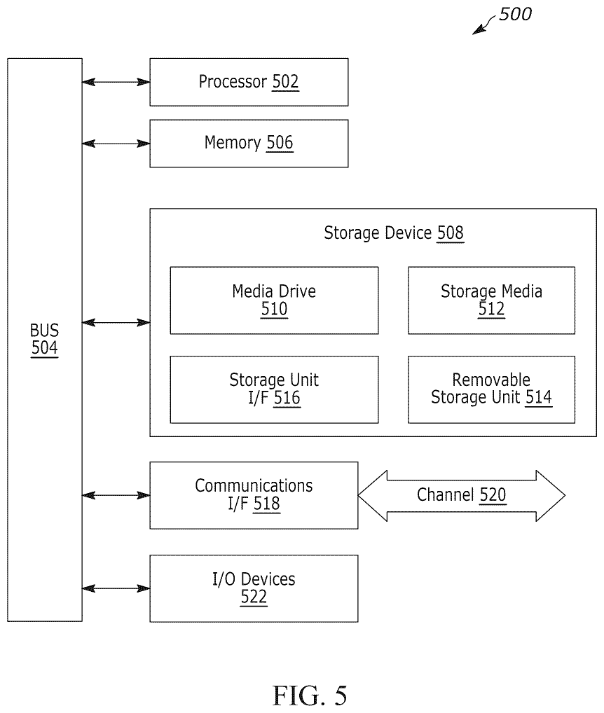

FIG. 5 illustrates an exemplary environment 500 suitable for implementing various embodiments of the present disclosure. The environment 500 includes a processor 502, a bus 504, a memory 506, storage devices 508, a media drive 510, storage media 512, a removable storage unit 514, a storage unit interface (I/F) 516, a communication interface (I/F) 518, a communication channel 520, and input/output (I/O) devices 522.

The processor 502 serves as the central processing unit (CPU) within the environment 500, enabling control over the execution of instructions for performing operations in accordance with the present disclosure. The processor 502 is communicatively coupled with the memory 506 and storage devices 508 via the bus 504, which facilitates data transfer and synchronization across these components. In this manner, the processor 502 accesses data stored in the memory 506, retrieves instructions, and directs communication among various elements of the environment 500. The environment 500 may include one or more processors, such as processor 502, implemented using a general-purpose or specialized processing engine, such as a microprocessor, microcontroller, or other control logic. In some embodiments, processor 502 may be an AI processor, implemented as a Tensor Processing Unit (TPU), graphical processing unit (GPU), or custom-programmable solution, such as a Field-Programmable Gate Array (FPGA).

The bus 504 enables an efficient data flow pathway that connects the processor 502 to the memory 506 and storage devices 508, as well as to the communication interface 518 and the I/O devices 522. The bus 504 operates as a high-speed data communication link, allowing for real-time data transfer and ensuring coordination between system components for executing complex computational tasks and data management.

The memory 506 serves as a primary, volatile storage medium, storing data and instructions currently accessed and used by the processor 502. This memory may include dynamic random-access memory (DRAM), static random-access memory (SRAM), and/or other high-speed memory types, enabling rapid access to instructions and data required by the processor 502 for executing the functionalities of the environment 500.

The storage devices 508 provide non-volatile, persistent data storage and include various sub-components such as the media drive 510, storage media 512, removable storage unit 514, and storage unit interface 516. The media drive 510 facilitates reading and writing of data on storage media 512, which may include hard drives, solid-state drives, optical discs, and other types of physical storage media. These components collectively store long-term data and software applications, supporting the computing environment's ability to retain information across sessions and shutdowns.

The removable storage unit 514 provides an option for inserting and removing portable storage media, allowing external data transfer and backup. This component is accessible via the storage unit I/F 516, which serves as an interface for managing data transfer between the processor 502 and removable storage media, enabling the environment 500 to expand storage capacity and facilitate data interchange with external devices or systems.

The communication I/F 518 provides a pathway for the environment 500 to interact with external networks and devices. This interface enables data exchange and connectivity, supporting protocols such as Ethernet, Wi-Fi, cellular networks, and other wired or wireless communication protocols. The communication I/F 518 operates through the channel 520, which facilitates external data exchange, allowing the environment 500 to interact seamlessly with remote servers, databases, and user devices for the transmission and reception of data in real time.

The I/O devices 522 represent an array of peripherals used for user interaction and system control, including input devices such as a keyboard, mouse, or touchscreen, as well as output devices such as a monitor or printer. These devices allow users to input data, receive feedback, and control operations within the environment 500, enhancing the user's ability to interact with and manage the computing system.

In various embodiments, the environment 500 may be configured to execute software applications, perform data processing tasks, and support data storage operations, while maintaining real-time communication with networked systems. This configuration enables the environment 500 to support multiple, concurrent operations essential for implementing the functionalities described in the present disclosure, with secure, flexible, and efficient data handling across internal and external channels.

The processor 502 may execute instructions stored in the memory 506 to perform the steps of the method described in FIG. 5, including creating the sensitive personal information, transmitting a copy of the sensitive personal information, receiving request requirements, verifying the request requirements, generating a binding challenge, verifying a binding proof generated and signed by the holder, generating a VC verified requirements, and transmitting the VC verified requirements to the holder. The communication I/F 518 may facilitate secure communication between the service provider, holder and issuer, ensuring that the sensitive data is protected during transmission. The I/O devices 522 may allow users to interact with the system, submit requests, and receive responses, while the storage devices 508 may store the sensitive data and system logs securely within the secured environment.

Thus, the disclosed method and system address the technical problem of securely verifying sensitive personal information in the decentralized ecosystem. The method and system utilize binding key pairs, Decentralized Identifiers (DIDs), and elliptic curve cryptography to enable users (holders) to verify their identity without exposing or storing sensitive personal data in centralized repositories. By offering users a secure, decentralized, and cryptographically verifiable means of identity validation, the system enhances trust in digital interactions while minimizing privacy risks associated with centralized data storage and potential breaches.

Thus, the disclosed method and system address the technical problem of designing thermostable protein variants without compromising the structural integrity or biological function of the protein. The method and system utilize a combination of structure-based masking strategies, evolutionary conservation analysis, and MPNNs to generate high-confidence mutant sequences. Additionally, the system incorporates structure prediction tools and classical molecular dynamics simulations to evaluate the thermal stability of the variants under dynamic conditions. The invention brings together information derived from both sequence-based features (such as MPNN outputs and TemStaPro stability predictions) and structure-based features (including conservation patterns and structural embeddings) to compute design and stability scores. The top-ranked variants are then re-evaluated using physics-based methods, including molecular dynamics simulation and free energy perturbation (FEP) analysis, to assess their dynamic behavior and functional retention under thermal stress. The selection and ranking of mutants are guided by qualifying criteria that account for both sequence-level plausibility and structural integrity. Furthermore, the system and method are designed to accommodate the incorporation of additional optimization objectives over time, such as improved pH stability, enhanced solubility, and reduced aggregation, without requiring major architectural changes. By reducing experimental load and enabling high-precision, modular protein design, the system offers a robust computational framework for accelerating the development of thermostable and functionally reliable protein variants for industrial and therapeutic applications.

In light of the above-mentioned advantages and the technical advancements provided by the disclosed method and system, the claimed steps as discussed above are not routine, conventional, or well understood in the art, as the claimed steps enable the following solutions to the existing problems in conventional technologies. Further, the claimed steps clearly bring an improvement in the functioning of the system itself as the claimed steps provide a technical solution to a technical problem.

It will be appreciated that, for clarity purposes, the above description has described embodiments of the invention with reference to different mechanical components and their functionalities. However, it will be apparent that any suitable distribution of functionality between different components or assemblies may be used without detracting from the invention. For example, functionality illustrated to be performed by separate components may be integrated into a single component or assembly. Hence, references to specific components are only to be seen as references to suitable means for providing the described functionality, rather than indicative of a strict structural or organizational configuration.

Although the present invention has been described in connection with some embodiments, it is not intended to be limited to the specific form set forth herein. Rather, the scope of the present invention is limited only by the claims. Additionally, although a feature may appear to be described in connection with particular embodiments, one skilled in the art would recognize that various features of the described embodiments may be combined in accordance with the invention.

Furthermore, although individually listed, a plurality of means, elements or process steps may be implemented by, for example, a single unit or processor. Additionally, although individual features may be included in different claims, these may possibly be advantageously combined, and the inclusion in different claims does not imply that a combination of features is not feasible and/or advantageous. Also, the inclusion of a feature in one category of claims does not imply a limitation to this category, but rather the feature may be equally applicable to other claim categories, as appropriate.

### TECHNICAL ADVANTAGES

The present disclosure described herein above has several technical advantages including, but not limited to:

Targeted Mutation Strategy: Improving the stability of a protein without compromising its function, this is achieved by eliminating mutations in critical conserved regions.

Structure-Aware Mutation Prediction: Incorporates 3D context and analyze higher likelihood of stabilizing effects. Uses evolutionary information and biophysical properties to score mutations.

MD Simulation Validation: Provides dynamic insight into protein behavior post-mutation. Verifies that predicted stabilizing mutations maintain structural integrity over time and under thermal stress. Captures local unfolding, RMSD/RMSF changes, and hydrogen bond stability.

Reduced Experimental Load: Narrows down mutation candidates before wet-lab testing, minimizes cost, time, and labor through computational pre-screening.

High Customizability: Can be tailored for different enzyme families or stability conditions (pH). Scalable with high-performance computing for large mutational libraries.

What is claimed is:

1. A system for generating thermostable variants of a protein, the system comprising:
a memory operatively associated with at least one processor, the memory including machine-executable instructions that, when executed by the at least one processor, cause the at least one processor to:
receive a three-dimensional structure of a target protein, wherein a plurality of mutable regions is identified within the three-dimensional structure, and wherein the plurality of mutable regions comprises solvent-exposed residues and loop regions;
perform a multiple sequence alignment of the target protein to identify a plurality of conserved residues in the target protein;
generate a fixed-position mask based on the plurality of conserved residues and a plurality of active site residues, wherein the fixed-position mask defines a set of excluded residues that are not subject to mutation;
generate a plurality of mutant protein sequences using a message-passing neural network by introducing mutations at unmasked positions within the plurality of mutable regions;
compute a design score for each of the plurality of mutant protein sequences, wherein the design score is based on Shannon entropy and log probability metrics calculated at each mutated position;
select a subset of the plurality of mutant protein sequences having design scores that satisfy a predefined threshold;
predict, for each mutant protein sequence in the selected subset, a corresponding three-dimensional structure;
compute a stability score for each predicted structure, wherein the stability score comprises one or more features selected from the group consisting of root mean square deviation (RMSD), predicted local distance difference test (pLDDT), solvent accessibility, amino acid composition, and structural embedding similarity; and
output a ranked list of thermostable mutant protein sequences based on the design score and the stability score.

2. The system of claim 1, wherein the at least one processor is further configured to:
perform a molecular dynamics simulation for each predicted structure under thermal stress conditions;
compute one or more dynamic simulation metrics for each predicted structure, wherein the dynamic simulation metrics comprise at least one of RMSD variation over time, radius of gyration, solvent-accessible surface area, and hydrogen bond retention; and
re-rank the selected mutant protein sequences based on the corresponding design score, the stability score, and the one or more dynamic simulation metrics.

3. The system of claim 1, wherein the solvent-exposed residues are identified using a neighbor search algorithm.

4. The system of claim 1, wherein the loop regions are inferred based on the spatial arrangement of atoms in the three-dimensional structure.

5. The system of claim 1, wherein the active site residues are determined based on proximity to a known ligand-binding region.

6. The system of claim 1, wherein the message-passing neural network is executed under multiple temperature parameters to simulate mutation generation at varying stringency levels.

7. The system of claim 6, wherein the temperature parameters include values selected from a group consisting of 0.1, 0.3, and 0.5.

8. The system of claim 1, wherein the design score is used to filter mutant protein sequences having a Shannon entropy less than 1.0 and a log probability greater than or equal to 0.5.

9. The system of claim 1, wherein the molecular dynamics simulations on the predicted structures evaluate the compactness of the corresponding three-dimensional structure of each mutant protein sequence using radius of gyration.

10. A computer-implemented method for generating thermostable variants of a protein, the method comprising:
receiving, by at least one processor, a three-dimensional structure of a target protein, wherein a plurality of mutable regions is identified within the three-dimensional structure, and wherein the plurality of mutable regions comprises solvent-exposed residues and loop regions;
performing, by the at least one processor, a multiple sequence alignment of the target protein to identify a plurality of conserved residues in the target protein;
generating, by the at least one processor, a fixed-position mask based on the plurality of conserved residues and a plurality of active site residues, wherein the fixed-position mask defines a set of excluded residues that are not subject to mutation;
generating, by the at least one processor, a plurality of mutant protein sequences using a message-passing neural network by introducing mutations at unmasked positions within the plurality of mutable regions;
computing, by the at least one processor, a design score for each of the plurality of mutant protein sequences, wherein the design score is based on Shannon entropy and log probability metrics calculated at each mutated position;
selecting, by the at least one processor, a subset of the plurality of mutant protein sequences having design scores that satisfy a predefined threshold;
predicting, for each mutant protein sequence in the selected subset, by the at least one processor, a corresponding three-dimensional structure;
computing, by the at least one processor, a stability score for each predicted structure based on one or more structural and sequence-based features; and
outputting, by the at least one processor, a ranked list of thermostable mutant protein sequences based on the design score and the stability score.

11. The method of claim 10, further comprising:
performing, by the at least one processor, molecular dynamics simulations on the predicted structures under thermal stress conditions;
computing, by the at least one processor, dynamic simulation metrics comprises at least one of: RMSD variation, radius of gyration, solvent-accessible surface area, and hydrogen bond retention; and
re-ranking, by the at least one processor, the thermostable mutant protein sequences based on the dynamic simulation metrics.

12. The method of claim 10, wherein the solvent-exposed residues are identified using a neighbor search algorithm.

13. The method of claim 10, wherein the loop regions are inferred based on the spatial arrangement of atoms in the three-dimensional structure.

14. The method of claim 10, wherein the active site residues are determined based on proximity to a known ligand-binding region.

15. The method of claim 10, wherein the message-passing neural network is executed under multiple temperature parameters to simulate mutation generation at varying stringency levels

16. The method of claim 10, wherein the molecular dynamics simulations on the predicted structures evaluate the compactness of the corresponding three-dimensional structure of each mutant protein sequence using radius of gyration.

17. The method of claim 10, wherein the molecular dynamics simulations on the predicted structures further evaluate local unfolding in the mutant structure over time.

18. The method of claim 10, wherein the fixed-position mask is dynamically generated based on evolutionary conservation scores derived from position-specific scoring matrices (PSSM).

19. The method of claim 10, wherein the at least one processor is further configured to prioritize mutations occurring in a hydrophobic core of the protein.

20. The method of claim 10, wherein the design score and stability score are combined using a machine learning model trained to identify high-stability protein variants, wherein the machine learning model is the message-passing neural network.
